(19) United States
(12) Patent Application Publication
(10) Pub. No.: US 2026/0237062 A1
(43) Pub. Date: Aug. 13, 2026

(54) ARTIFICIAL INTELLIGENCE SYSTEMS AND RELATED METHODS FOR THE DETECTION OF DISEASE STATES USING URINE-RELATED IMAGE DATA

(71) Applicant: THE JOHNS HOPKINS UNIVERSITY, Baltimore, MD (US)

(72) Inventors: Therese L. CANARES, Baltimore, MD (US); Mathias UNBERATH, Baltimore, MD (US)

(21) Appl. No.: 19/154,662

(22) PCT Filed: Feb. 8, 2024
(86) PCT No.: PCT/US2024/014930

§ 371 Date: Aug. 7, 2025

Related U.S. Application Data
(60) Provisional application No. 63/484,022, filed on Feb. 9, 2023.

(51) Int. Cl. G06T 7/00; G06T 15/20; G06V 10/764; G16H 50/20

(52) U.S. Cl. CPC G06T 7/0012; G06T 15/205; G06V 10/764; G16H 50/20; G06T 2207/20084

(57) ABSTRACT

Examples may provide computer-implemented methods of generating prediction scores for disease states in test subjects. In some embodiments, the methods include passing a set of features extracted from urine-related image data obtained from a given test subject through an artificial intelligence (AI) algorithm that is configured to generate prediction scores for the disease state in subjects based at least in part on the set of features extracted from the urine-related image data. In some embodiments, the methods also include outputting from the AI algorithm a prediction score for the disease state in the given test subject indicated by the set of features extracted from the urine-related image data obtained from the given test subject. Additional methods and related systems are also provided herein.

### CROSS-REFERENCE TO RELATED APPLICATIONS

This application is the national stage entry of International Patent Application No. PCT/US2024/014930, filed on Feb. 8, 2024, and published as WO 2024/168107 A1 on Aug. 15, 2024, which claims the benefit of U.S. Provisional Patent Application Ser. No. 63/484,022, filed Feb. 9, 2023, which are hereby incorporated by reference herein in their entireties.

### FIELD

This disclosure relates generally to artificial intelligence, e.g., in the context of medical applications, such as pathology.

### BACKGROUND

Urinary tract infections (UTIs) are among the most common reasons patients seek acute medical care. In 2021, for example, Johns Hopkins Medicine diagnosed over 51,000 patients with a UTI. UTIs are generally diagnosed with a urinalysis, confirmed with a urine culture, and treated with oral antibiotics.

Telehealth is an option for UTI care. Telehealth involves the use of digital information and communication technologies to access health care services from locations that are remote from healthcare providers, such as from the patient's home. The communication technologies often include mobile devices, such as smartphones and tablet computers. In the case of UTIs, telehealth doctors may prescribe antibiotics for typical urinary symptoms without a urinalysis. However, up to 80% of patients can have atypical symptoms and the pre-test probability of UTI from symptoms alone is only 73% at best. A urinalysis or culture is important to differentiate a UTI from other conditions, but urinalyses are generally not available at home, during a telehealth visit.

To date, machine learning and medical image analysis for detection of UTIs has not been optimized for patient use with a smartphone or other mobile device. Past work has focused on laboratory samples, such as images of urine that are microscopic, plated on petri dishes, or use infrared spectroscopy to identify strains of drug resistant bacteria. Other studies used inputs of demographic data, historical risk factors, or urine lab results into their machine learning models to predict UTI. Microscopic images and laboratory data are not feasible to collect at the point-of-care during a telehealth visit.

Accordingly, it is apparent that there is a need for additional methods of detecting disease states, such as UTIs, including from locations that are remote from the patient using urine sample image data.

### SUMMARY

The present disclosure provides, in certain aspects, an artificial intelligence (AI) system capable of generating prediction scores for disease states in test subjects using urine-related image data. In some aspects, for example, the present disclosure provides a computational framework for generating prediction scores for urinary tract infections (UTIs) or other urinary tract-related disease states in test subjects that uses electronic neural networks that have been trained with features extracted from images of urine samples obtained from reference subjects. In some embodiments, the methods and systems of the present disclosure predict measurements of various markers of kidney function, including blood urea nitrogen (BUN) and/or creatinine, among others. In some embodiments, patients suspected of having a urinary tract-related disease state can receive a diagnostic test at home, for example, by uploading a picture or video of their urine sample using a mobile device, and the analysis of the uploaded data is performed by the computer program products and related systems disclosed herein. These and other aspects will be apparent upon a complete review of the present disclosure, including the accompanying figures.

According to various embodiments, a computer-implemented method of generating a prediction score for a disease state in a test subject is presented. The method includes: passing a data set that comprises one or more features extracted from urine-related image data obtained from a test subject through an artificial intelligence (AI) algorithm that is trained to generate one or more prediction scores for the disease state in subjects based at least in part on the features extracted from the urine-related image data; and outputting from the AI algorithm a prediction score for the disease state in the test subject indicated by the data set obtained from the test subject.

According to various embodiments, computer-implemented method of generating a prediction score for a disease state in a test subject is presented. The method includes: receiving, by a computer, a data set that comprises urine-related image data obtained from a test subject and/or one or more features extracted from the urine-related image data; passing, by the computer, at least a portion of the data set through an artificial intelligence (AI) algorithm that is trained to generate one or more prediction scores for the disease state in subjects based at least in part on one or more portions of the data set; and outputting, by the computer, from the AI algorithm a prediction score for the disease state in the test subject indicated by at least the portion of the data set passed through the AI algorithm.

Various optional features of the above embodiments include the following. The AI algorithm has been trained on a first set of training data that comprises a plurality of sets of features extracted from urine-related image data obtained from reference subjects, wherein the urine-related image data obtained from the reference subjects are each labeled with a positive or negative disease state ground truth classification for a given reference subject, and wherein one or more predictions for a positive or negative disease state classification for the given reference subject are made based on the urine-related image data obtained from the given reference subject, which predictions are compared to the ground truth classification for the given reference subject when the AI algorithm is trained. The AI algorithm comprises a machine learning algorithm, an electronic neural network, and/or a deep learning algorithm. The method includes generating a therapy recommendation for the test subject based upon the prediction score output from the AI algorithm. The method includes administering a therapy to the test subject based upon the prediction score output from the AI algorithm. The prediction score comprises a classification score. The prediction score comprises a regression score.

Various additional optional features of the above embodiments include the following. The AI algorithm is trained to generate prediction scores for the disease state in the subjects based at least in part on a set of features extracted from other types of data related to the subjects, and wherein the computer-implemented method further comprises passing a set of features extracted from the other types of data related to the test subject through the AI algorithm. The AI algorithm has been trained on a second set of training data that comprises a plurality of sets of features extracted from the other types of data related to the reference subjects. The AI algorithm comprises at least first and second parts, wherein the first part is trained on a plurality of sets of features extracted from the urine-related image data related to the reference subjects, and wherein the second part is trained on a plurality of sets of features extracted from the other types of data related to the reference subjects. The other types of data comprise demographic data, symptom data, risk factor data, physical examination data, or a combination thereof. The demographic data comprises one or more of: subject age and subject sex. The symptom data comprises one or more subject symptoms selected from the group consisting of: painful urination, urinary frequency, urinary urgency, fever, abdominal pain, vomiting, back pain, flank pain, change in the appearance of the urine, vaginal itching, penile itching, vaginal discharge, penile discharge, number of days with symptoms, malodorous urine, incontinence, change in behavior, and change in mental status. The risk factor data comprises one or more subject risk factors selected from the group consisting of: circumcision status, urologic conditions, neurologic conditions, renal conditions, and prior history of urinary tract infection. The disease state comprises a urinary tract infection. The disease state comprises a bacterial infection, a viral infection, a fungal infection, a parasitic infection, hematuria (blood in the urine), proteinuria (protein in the urine), pyuria, glucosuria, or dehydration. The bacterial infection comprises a Gonorrhea infection, a Chlamydia infection, or a combination thereof. The viral infection comprises a herpes simplex virus (HSV) infection, a human immunodeficiency virus (HIV) infection, a human papilloma virus (HPV) infection, or a combination thereof. The fungal infection comprises a Candida infection. The parasitic infection comprises a Trichomonas infection, a schistosome infection, a filarial worm infection, or a combination thereof.

Various additional optional features of the above embodiments also include the following. The prediction score comprises a probability of a positive or negative disease state classification for the test subject. The urine-related image data comprises one or more images of urine samples obtained from the test and reference subjects. The images of urine samples obtained from the test and reference subjects are not magnified using a microscope. The method includes identifying at least one region of interest from one or more images of the urine samples obtained from the test and reference subjects. The method includes generating a three-dimensional (3D) model of the region of interest from the images of the urine samples. The method includes generating one or more rendered images from the 3D model. The method includes standardizing the rendered images. The method includes generating an estimated volume of the region of interest from the 3D model. The first set of training data comprises the rendered images and/or the estimated volume of the region of interest. The images of urine samples obtained from the test and reference subjects are obtained from one or more videos of the urine samples obtained from the test and reference subjects. The images of urine samples obtained from the test and reference subjects can include, for example, still images, a series of still images, videos, and the like. The videos of the urine samples each comprise multiple views of the urine samples disposed in sample containers or other liquid receptacles (e.g., images or videos of urine samples disposed in a toilet, etc.). The method further includes filtering frames of the videos using a frame selection process that comprises separating a given video into individual frames, selecting one or more of the individual frames having a specified quality level to produce a set of selected frames, and generating one or more bounding boxes that enclose one or more areas of interest in one or more frames in the set of selected frames to produce a set of designated areas of interest. The method further includes pre-processing the videos using a process that comprises automatically isolating selected frames from the videos to produce a set of selected frames, automatically generating one or more bounding boxes that enclose one or more areas of interest in one or more frames in the set of selected frames, and automatically extracting coordinates of the bounding boxes to produce a set of isolated areas of interest. The method further includes labeling a given area of interest in the set of designated areas of interest with a label that indicates a content of the given area of interest. The method includes obtaining the images of the urine samples using a mobile device. The test subject obtains the images of the urine samples. A healthcare provider obtains the images of the urine samples.

Various additional optional features of the above embodiments further include the following. The features comprise numerical vectors. The AI algorithm has been further trained on a second set of training data that comprises a plurality of sets of features extracted from numerical vectors representing sets of parameterized symptoms from the reference subjects and wherein the computer-implemented method further comprises passing a set of features extracted from a numerical vector representing a set of parameterized symptoms from the test subject through the AI algorithm. The numerical vectors representing the set of parameterized symptoms from the reference subjects and from the test subject each comprise at least a 15-dimensional vector. The method further includes mapping the features of the data set to a bidimensional vector that corresponds to the prediction score for the disease state in the test subject. The AI algorithm uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

According to various embodiments, a system for generating a prediction score for a disease state in a test subject using an artificial intelligence (AI) algorithm is presented. The system includes a processor; and a memory communicatively coupled to the processor, the memory storing instructions which, when executed on the processor, perform operations including: passing a data set that comprises one or more features extracted from urine-related image data obtained from a test subject through the AI algorithm that is trained to generate one or more prediction scores for the disease state in subjects based at least in part on the first set of features extracted from the urine-related image data; and outputting from the AI algorithm a prediction score for the disease state in the test subject indicated by the data set obtained from the test subject.

Various optional features of the above embodiments include the following. The instructions which, when executed on the processor, further perform operations comprising: receiving the urine-related image data obtained from the test subject and/or one or more features extracted from the urine-related image data. The instructions which, when executed on the processor, further perform operations comprising: extracting the features from the urine-related image data. The instructions which, when executed on the processor, further perform operations comprising: generating a therapy recommendation for the test subject based upon the prediction score output from the AI algorithm. The AI algorithm has been trained on a first set of training data that comprises a plurality of sets of features extracted from urine-related image data obtained from reference subjects, wherein the urine-related image data obtained from the reference subjects are each labeled with a positive or negative disease state ground truth classification for a given reference subject, and wherein one or more predictions for a positive or negative disease state classification for the given reference subject are made based on the urine-related image data obtained from the given reference subject, which predictions are compared to the ground truth classification for the given reference subject when the AI algorithm is trained. The AI algorithm comprises a machine learning algorithm, an electronic neural network, and/or a deep learning algorithm. The prediction score comprises a classification score. The prediction score comprises a regression score.

Various additional optional features of the above embodiments include the following. The AI algorithm is trained to generate prediction scores for the disease state in the subjects based at least in part on a set of features extracted from other types of data related to the subjects, and wherein the instructions which, when executed on the processor, further perform operations comprising: passing a set of features extracted from the other types of data related to the test subject through the AI algorithm. The AI algorithm has been trained on a second set of training data that comprises a plurality of sets of features extracted from the other types of data related to the reference subjects. The AI algorithm comprises at least first and second parts, wherein the first part is trained on a plurality of sets of features extracted from the urine-related image data related to the reference subjects, and wherein the second part is trained on a plurality of sets of features extracted from the other types of data related to the reference subjects. The other types of data comprise demographic data, symptom data, risk factor data, physical examination data, or a combination thereof. The demographic data comprises one or more of: subject age and subject sex. The symptom data comprises one or more subject symptoms selected from the group consisting of: painful urination, urinary frequency, urinary urgency, fever, abdominal pain, vomiting, back pain, flank pain, change in the appearance of the urine, vaginal itching, penile itching, vaginal discharge, penile discharge, number of days with symptoms, malodorous urine, incontinence, change in behavior, and change in mental status. The risk factor data comprises one or more subject risk factors selected from the group consisting of: circumcision status, urologic conditions, neurologic conditions, renal conditions, and prior history of urinary tract infection. The disease state comprises a urinary tract infection. The disease state comprises a bacterial infection, a viral infection, a fungal infection, a parasitic infection, hematuria, proteinuria, pyuria, glucosuria, or dehydration. The bacterial infection comprises a Gonorrhea infection, a Chlamydia infection, or a combination thereof. The viral infection comprises a herpes simplex virus (HSV) infection, a human immunodeficiency virus (HIV) infection, a human papilloma virus (HPV) infection, or a combination thereof. The fungal infection comprises a Candida infection. The parasitic infection comprises a Trichomonas infection, a schistosome infection, a filarial worm infection, or a combination thereof.

Various additional optional features of the above embodiments also include the following. The prediction score comprises a probability of a positive or negative disease state classification for the test subject. The urine-related image data comprises one or more images of urine samples obtained from the test and reference subjects. The images of urine samples obtained from the test and reference subjects are not magnified using a microscope. The instructions which, when executed on the processor, further perform operations comprising: generating a three-dimensional (3D) model of the region of interest from the images of the urine samples. The instructions which, when executed on the processor, further perform operations comprising: generating one or more rendered images from the 3D model. The instructions which, when executed on the processor, further perform operations comprising: standardizing the rendered images. The instructions which, when executed on the processor, further perform operations comprising: generating an estimated volume of the region of interest from the 3D model. The first set of training data comprises the rendered images and/or the estimated volume of the region of interest. The images of urine samples obtained from the test and reference subjects are obtained from one or more videos of the urine samples obtained from the test and reference subjects. The videos of the urine samples each comprise multiple views of the urine samples disposed in sample containers or other liquid receptacles. The instructions which, when executed on the processor, further perform operations comprising: filtering frames of the videos using a frame selection process that comprises separating a given video into individual frames; selecting one or more of the individual frames having a specified quality level to produce a set of selected frames; and generating one or more bounding boxes that enclose one or more areas of interest in one or more frames in the set of selected frames to produce a set of designated areas of interest. The instructions which, when executed on the processor, further perform operations comprising: isolating selected frames from the videos to produce a set of selected frames; generating one or more bounding boxes that enclose one or more areas of interest in one or more frames in the set of selected frames; and extracting coordinates of the bounding boxes to produce a set of isolated areas of interest. The instructions which, when executed on the processor, further perform operations comprising: labeling a given area of interest in the set of designated areas of interest with a label that indicates a content of the given area of interest.

Various additional optional features of the above embodiments further include the following. The features comprise numerical vectors. The AI algorithm has been further trained on a second set of training data that comprises a plurality of sets of features extracted from numerical vectors representing sets of parameterized symptoms from the reference subjects and wherein the computer-implemented method further comprises passing a set of features extracted from a numerical vector representing a set of parameterized symptoms from the test subject through the AI algorithm. The numerical vectors representing the set of parameterized symptoms from the reference subjects and from the test subject each comprise at least a 15-dimensional vector. The instructions which, when executed on the processor, further perform operations comprising: mapping the features of the data set to a bidimensional vector that corresponds to the prediction score for the disease state in the test subject. The AI algorithm uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

### DRAWINGS

The above and/or other aspects and advantages will become more apparent and more readily appreciated from the following detailed description of examples, taken in conjunction with the accompanying drawings, in which:

FIG. 1A is a flow chart that schematically shows exemplary method steps of generating a prediction score for a disease state in a test subject according to some aspects disclosed herein.

FIG. 1B is a flow chart that schematically shows exemplary method steps of generating a prediction score for a disease state in a test subject according to some aspects disclosed herein.

FIG. 2 is a schematic diagram of an exemplary system suitable for use with certain aspects disclosed herein.

FIG. 3 is a schematic diagram of an exemplary image-based classifier suitable for use with certain aspects disclosed herein.

FIG. 4 is a schematic diagram of an exemplary clinical decision support system suitable for use with certain aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Artificial Intelligence Algorithm: As used herein, “artificial intelligence algorithm” or “AI algorithm” refers to a set of computer instructions that takes in training or input data that assists the algorithm to learn to operate on its own. Data used by an AI algorithm can include various data formats, including unstructured or qualitative data, structured or quantitative data, or semi-structured data. Machine learning and deep learning algorithms are types of AI algorithms that generate expert systems for making predictions or classifications based on the input data.

Classifier As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like).

Labeled: As used herein, “labeled” in the context of data sets or points refers to data that is classified as, or otherwise associated with, having or lacking a given characteristic or property.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.” Data used by a machine learning algorithm is typically structured data or semi-structured data.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

Value: As used herein, “value” generally refers to an entry in a dataset that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. INTRODUCTION

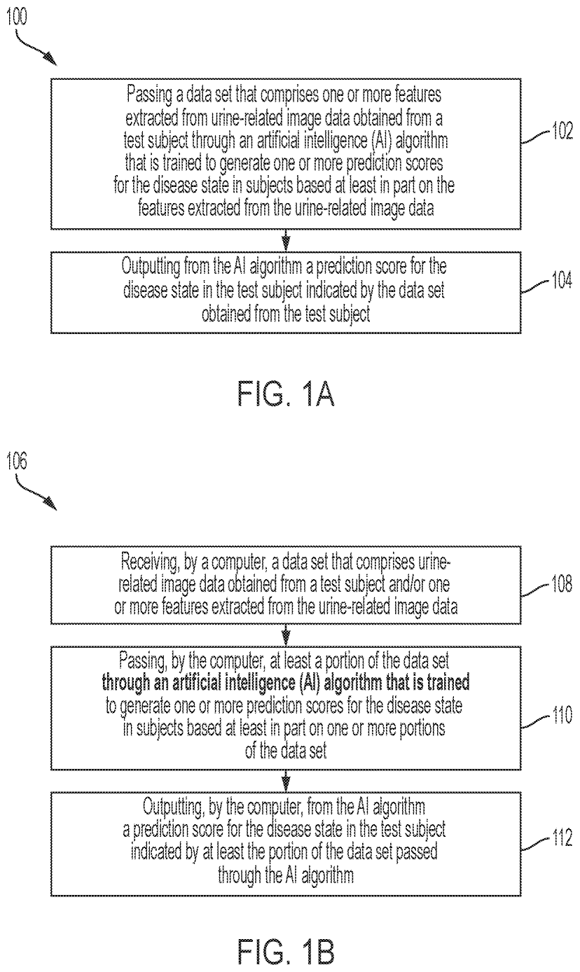

In some aspects, the present disclosure provides computer-implemented methods of generating a prediction score (e.g., a classification score, a regression score, etc.) for a disease state in a test subject. To illustrate, FIG. 1A is a flow chart that schematically shows certain of these exemplary method steps. As shown, method 100 includes passing a data set that comprises one or more features extracted from urine-related image data obtained from a test subject through an artificial intelligence (AI) algorithm that is trained to generate one or more prediction scores for the disease state in subjects based at least in part on the features extracted from the urine-related image data (step 102). Method 100 also includes outputting from the AI algorithm a prediction score for the disease state in the test subject indicated by the data set obtained from the test subject (step 104).

To further illustrate, FIG. 1B is a flow chart that schematically shows some additional exemplary method steps. As shown, method 106 includes receiving, by a computer, a data set that comprises urine-related image data obtained from a test subject and/or one or more features extracted from the urine-related image data (step 108). Method 106 also includes passing, by the computer, at least a portion of the data set through an artificial intelligence (AI) algorithm that is trained to generate one or more prediction scores for the disease state in subjects based at least in part on one or more portions of the data set (step 110). In addition, method 106 further includes outputting, by the computer, from the AI algorithm a prediction score for the disease state in the test subject indicated by at least the portion of the data set passed through the AI algorithm (step 112).

In some embodiments, the AI algorithm has been trained on a first set of training data that includes a plurality of sets of features extracted from urine-related image data obtained from reference subjects. In some embodiments, the urine-related image data obtained from the reference subjects are each labeled with a positive or negative disease state ground truth classification for a given reference subject. Typically, a prediction for a positive or negative disease state classification for the given reference subject is made based on the urine-related image data obtained from the given reference subject. Such predictions are generally compared to the ground truth classification for the given reference subject when the AI algorithm is trained. The AI algorithm is typically a machine learning algorithm, an electronic neural network, and/or a deep learning algorithm as described further herein. In some embodiments, the urine-related image data obtained from the test subject is received by a system that comprises the AI algorithm from a location that is remote from the system, such as from the test subject's home (e.g., as part of a telehealth visit or the like). In some embodiments, a prediction score includes a probability of a positive or negative disease state classification for the test subject.

Typically, the features extracted from the urine-related image data obtained from the test and reference subjects comprise numerical vectors. In some embodiments, the AI algorithm has been further trained on a second set of training data that comprises a plurality of sets of features extracted from numerical vectors representing sets of parameterized demographic data, symptom data, risk factor data, and/or physical examination data from the reference subjects. In these embodiments, methods 100 and 106 also generally include passing a set of features extracted from a numerical vector representing a set of parameterized demographic data, symptom data, risk factor data, and/or physical examination data from the test subject through the AI algorithm to generate a prediction score for a given disease state in the test subject. In some embodiments, the numerical vectors from the reference subjects and from the test subject each comprise at least a 15-dimensional vector (e.g., about a 20-dimensional vector, about a 25-dimensional vector, about a 30-dimensional vector, about a 35-dimensional vector, about a 40-dimensional vector, about a 45-dimensional vector, about a 50-dimensional vector, about a 60-dimensional vector, about a 70-dimensional vector, about an 80-dimensional vector, about a 90-dimensional vector, about a 100-dimensional vector, or more dimensional vector). In some embodiments, methods 100 and 106 further include mapping the features of the data set to a bidimensional vector that corresponds to the prediction score for the disease state in the test subject. In some embodiments, the AI algorithm uses one or more algorithms selected from, for example, a random forest algorithm, a support vector machine algorithm, a decision tree algorithm, a linear classifier algorithm, a logistic regression, a linear regression algorithm, a polynomial regression algorithm, or the like.

In some embodiments, methods 100 and 106 also include generating a further diagnostic recommendation (e.g., to perform additional laboratory tests to confirm an indicated diagnosis (e.g., confirmatory testing via cell culture, rapid antigen testing, or other molecular diagnostic testing) and/or to further stratify a given diagnostic determination) for the test subject based upon the prediction score output from the AI algorithm. In some embodiments, methods 100 and 106 also include generating a therapeutic/clinical recommendation for the test subject based upon the prediction score output from the AI algorithm, such as when there is a positive indication of the presence of the disease state (e.g., an infectious disease state, a cancerous disease state, or the like) in the test subject. In some of these embodiments, methods 100 and 106 further include administering a therapy (e.g., an antibiotic therapy, an antifungal therapy, an immunological therapeutic agent, a symptomatic therapy (e.g., an analgesic administration), a chemotherapeutic agent, a radiotherapy, a surgical intervention (e.g., incision and drainage, etc.), and/or the like) to the test subject based upon the prediction score output from the AI algorithm. As used herein, “administer” or “administering” a therapeutic agent to a subject means to give, apply or bring the composition comprising the therapeutic agent into contact with the subject. Such administration can be accomplished by any of a number of routes, including, for example, topical, oral, subcutaneous, intramuscular, intraperitoneal, intravenous, intrathecal and intradermal. In some embodiments, a given test subject is referred to a specific healthcare provider or specialist for further evaluation and/or treatment based, at least in part, upon the prediction score output from the AI algorithm. In some embodiments, infection control or isolation procedures are triggered when a given prediction score is output from the AI algorithm. In some embodiments, methods 100 and 106 further include discontinuing administering a therapy to the test subject based upon the prediction score output from the AI algorithm.

In some embodiments, the AI algorithm is trained to generate prediction scores for the disease state in the subjects based at least in part on a set of features extracted from other types of data related to the subjects. In these embodiments, methods 100 and 106 typically further include passing a set of features extracted from the other types of data related to the test subject through the AI algorithm. In these embodiments, the AI algorithm has been trained on a second set of training data that comprises a plurality of sets of features extracted from the other types of data related to the reference subjects. In some embodiments, the AI algorithm comprises at least first and second parts, in which the first part is trained on a plurality of sets of features extracted from the urine-related image data related to the reference subjects, and in which the second part is trained on a plurality of sets of features extracted from the other types of data related to the reference subjects. Urine-related image data as well as other types of data used in the methods of the present disclosure are described further herein.

The other types of data include, for example, demographic data, symptom data, risk factor data, physical examination data, or a combination thereof. The demographic data can include subject age and subject sex. The symptom data can include subject symptoms, such as painful urination, urinary frequency, urinary urgency, fever, abdominal pain, vomiting, back pain, flank pain, change in the appearance of the urine, vaginal itching, penile itching, vaginal discharge, penile discharge, number of days with symptoms, malodorous urine, incontinence, change in behavior, and change in mental status. The risk factor data can include subject risk factors, such as circumcision status, urologic conditions, neurologic conditions, renal conditions, and prior history of urinary tract infection.

In some embodiments, the disease state comprises a urinary tract infection. In some embodiments, the disease state is a bacterial infection, a viral infection, a fungal infection, a parasitic infection, hematuria (blood in the urine), proteinuria (protein in the urine), pyuria, glucosuria, or dehydration. The bacterial infection can include, for example, a Gonorrhea infection, a Chlamydia infection, or a combination thereof. The viral infection can include, for example, a herpes simplex virus (HSV) infection, a human immunodeficiency virus (HIV) infection, a human papilloma virus (HPV) infection, or a combination thereof. The fungal infection can include, for example, a Candida infection. The parasitic infection can include, for example, a Trichomonas infection, a schistosome infection, a filarial worm infection, or a combination thereof.

In some embodiments, the urine-related image data utilized in the methods disclosed herein include images of urine samples (e.g., in urine sample containers) obtained from the test and reference subjects. In some embodiments, the images of urine samples obtained from the test and reference subjects are not magnified using a microscope. In some embodiments, methods 100 and 106 include identifying regions of interest from images of the urine samples obtained from the test and reference subjects. In some embodiments, methods 100 and 106 include generating a three-dimensional (3D) model of the region of interest from the images of the urine samples. In some of these embodiments, the methods include generating one or more rendered images from the 3D model, which renderings are optionally standardized. In some embodiments, the methods include generating an estimated volume of the region of interest from the 3D model. Optionally, the first set of training data comprises the rendered images and/or the estimated volume of the region of interest.

The images of urine samples obtained from the test and reference subjects can include, for example, still images, a series of still images, videos, and the like. In some embodiments, the images of urine samples obtained from the test and reference subjects are obtained from one or more videos of the urine samples obtained from the test and reference subjects. In some of these embodiments, the videos of the urine samples each comprise multiple views of the urine samples disposed in sample containers or other liquid receptacles (e.g., images or videos of urine samples disposed in a toilet, etc.). In some embodiments, the methods further include filtering frames of the videos using a frame selection process that includes separating a given video into individual frames, selecting one or more of the individual frames having a specified quality level to produce a set of selected frames, and generating one or more bounding boxes that enclose one or more areas of interest in one or more frames in the set of selected frames to produce a set of designated areas of interest. In some embodiments, the methods further include pre-processing the videos using a process that comprises automatically isolating selected frames from the videos to produce a set of selected frames, automatically generating one or more bounding boxes that enclose one or more areas of interest in one or more frames in the set of selected frames, and automatically extracting coordinates of the bounding boxes to produce a set of isolated areas of interest. In some embodiments, the methods further include labeling a given area of interest in the set of designated areas of interest with a label that indicates a content of the given area of interest. In some embodiments, methods 100 and 106 include obtaining the images of the urine samples using a mobile device. In some of these embodiments, the test subject obtains the images of the urine samples, whereas in other embodiments, a healthcare provider or other third-party obtains the images of the urine samples.

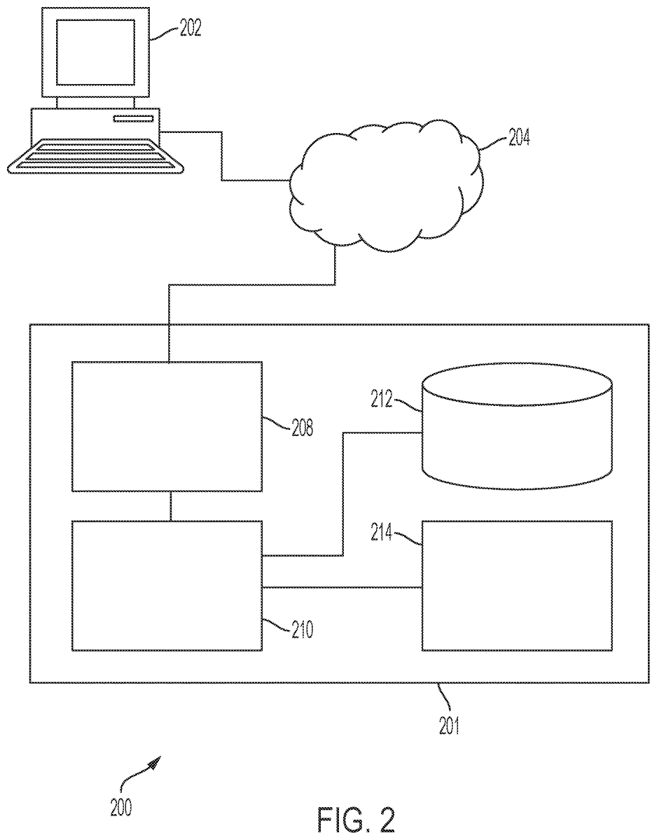

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of urine-related image or other data suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. DESCRIPTION OF EXAMPLE EMBODIMENTS

### Example: A Method to Identify Urinary Tract Infections Using Smartphone Images and Artificial Intelligence

### Introduction:

Urinary tract infections, or cystitis are extremely common in healthy females. Factors that increase risk of urinary tract infections can include neurogenic bladder, underlying urologic abnormalities, immunocompromised states, young or old age, and pregnancy. These factors can also complicate an individual's mobility, transportation, or feasibility to seek medical care.

When patients see a healthcare provider for urinary symptoms, they often send a urinalysis or urine culture to test for a urinary tract infection. Patients who have challenges to seek in-person medical care may opt for a telehealth visit. However, the nature of telehealth precludes the ability to perform point of care urine testing with an on-demand, unscheduled healthcare visit.

Currently, healthcare providers are weary to treat a urinary tract infection by telehealth, due to the inability to send urine testing and the risk of missing an alternate diagnosis. There is an apparent need for additional methods to test and risk stratify patients with urinary symptoms in a remote setting.

While studies describe deep learning to analyze urine sediment at a microscopic level, there is no literature on machine learning applied to macro, digital images of urine specimens to screen for UTI.

Different deep learning algorithms and convolutional architectures (CNN) have been proposed for general classification tasks. CNNs architectures, like DenseNet3 and ResNet4 have made great progress for medical image classification tasks.

In this example, we describe a classifier that uses deep learning techniques to identify positive and negative samples for urinary tract infections using the images extracted from a video recording of a urine sample.

### Dataset:

### Data Acquisition:

There are 252 urine sample videos (26 positives, 226 negatives). Each video corresponds to a patient. Videos were recorded using a consumer smartphone camera.

Additional features recorded include demographic data, symptom data, or a combination thereof.

Demographic data comprises one or more of: subject age, sex.

Symptoms that can include painful urination, urinary frequency, urinary urgency, fever, abdominal pain, vomiting, back pain, change in the appearance of the urine, vaginal itching (females) or vaginal discharge (females), and number of days with symptoms.

Additional risk factors include circumcision status (males), urologic conditions, neurologic conditions, and prior history of urinary tract infection.

The disease state comprises positive growth from a urine culture, including a bacterial infection, yeast infection, gonorrhea infection, chlamydia infection, or a combination thereof. Disease state also includes urinalysis status such as hematuria, proteinuria, pyuria, glucosuria, or dehydration.

### Method:

### Video Pre-Processing:

The video pre-processing is employed to generate a set of good-quality still frames for training and validating the AI models. The process starts with video acquisition. The video contains a scene of the urine sample under different views or angles. Different frames might not be suitable for model training and evaluation (e.g., due to lighting or obscuring of the urine sample by the borders of the container). Hence, we employ a frame selection process in order to filter the frames.

The frame selection utilizes a computational program that allows a user to visualize and select/confirm the frames to be employed for training the model. First, a computational process separates the video into individual frames. The user can visualize these frames to select the ones that present a good quality for model training. Then, the user can employ a drawing tool to generate a bounding box enclosing the area of interest inside the image. The region of interest can contain the urine sample and the borders of the container, and any other element of the still frame that could contribute to the learning process of the model. Each region of interest is associated with a label that indicates its content. Once all the desired frames and regions of interest are selected, the information is saved in a text format suitable to be easily read by the learning algorithm.

Ten frames are extracted and annotated from every video. The annotation is a bounding box of the region of interest manually added. In order to evaluate the methods, we employed 5-fold cross-validation.

The dataset is split into 5 subsets with the same size to conduct 5-fold cross-validation. We used one subset as the validation set and the remaining four as the training set within a fold. We select different training/validation subsets for each fold, so that all parts have been validated after 5-fold cross-validation. The proportion of positive and negative samples remains the same in the training set and validation set, which is 10% positive, vs 90% negative.

### Deep Learning Techniques:

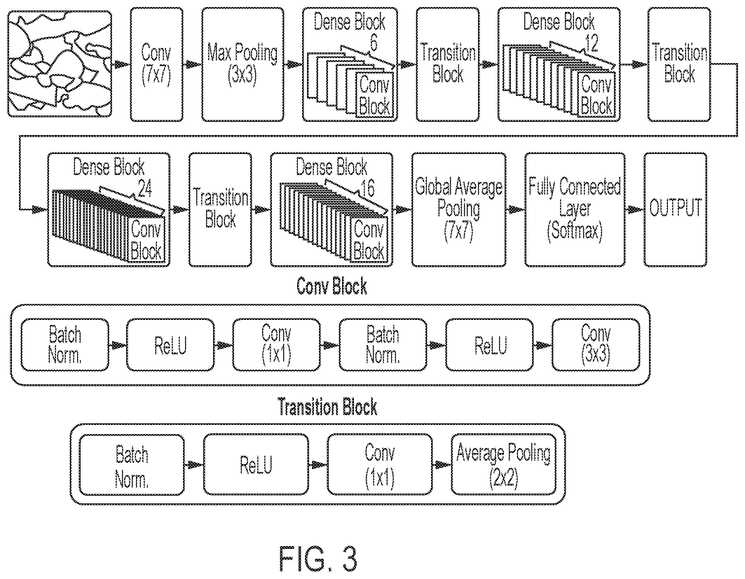

In this example, we developed a DenseNet-based classification framework for urinary tract infection identification. We employed the model DenseNet121 (FIG. 3) provided by PyTorch as the pretrained on binary classification model and fine-tune it for our task. The loss function is Focal Loss7. During each fold of the cross-validation, we trained the model for 10 epochs. For the training set images, data augmentation methods (horizontal flip, random crop, and random rotation) were performed.

### Results:

TABLE 1
The metrics of the validation set at the 10th epoch of each fold

| | | | Positive | Negative | |
|---|---|---|---|---|---|
| | | | Predictive | Predictive | |
| | Sensitivity | Specificity | Value | Value | AUC |
| Fold 1 | 4.00% | 96.12% | 10.53% | 90.15% | 0.7602 |
| Fold 2 | 69.57% | 91.21% | 44.44% | 96.74% | 0.8807 |
| Fold 3 | 34.78% | 95.19% | 43.24% | 93.27% | 0.9114 |
| Fold 4 | 44.00% | 87.70% | 28.57% | 93.33% | 0.8384 |
| Fold 5 | 35.00% | 90.04% | 31.82% | 90.85% | 0.7074 |
| Average | 37.47% | 92.05% | 31.72% | 92.87% | 0.8192 |

### User Workflow:

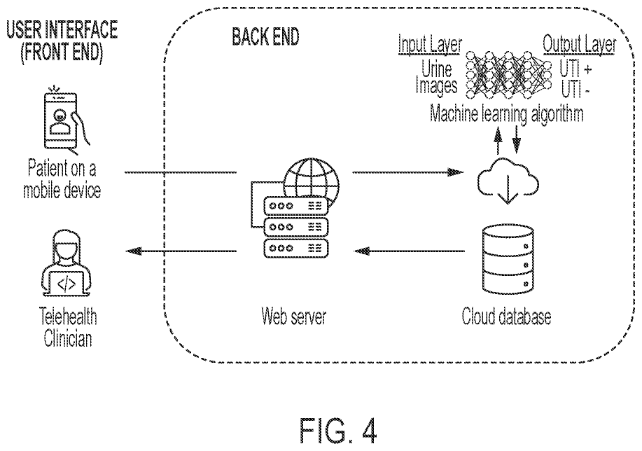

FIG. 4 Illustrates an exemplary user workflow for the UTI-AI application for telehealth. A patient with urinary symptoms will request a telehealth visit. Key words in the chief complaint or symptoms will prompt a request for the patient to record and upload a video of their urine sample. This video will be transmitted to a cloud database, where the AI system will extract key frames, segment the region of interest, then analyze the image using the predictive model. The output will be a positive or negative prediction for urinary tract infection (or other disease states). That prediction is transmitted to both the patient and telehealth clinician. The telehealth clinician uses the prediction to guide their medical decision making. In some embodiments, workflows of the present disclosure do not involve clinicians and instead are implemented as direct to consumer applications.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A computer-implemented method of generating a prediction score for a disease state in a test subject, the method comprising:
passing a data set that comprises one or more features extracted from urine-related image data obtained from a test subject through an artificial intelligence (AI) algorithm that is trained to generate one or more prediction scores for the disease state in subjects based at least in part on the features extracted from the urine-related image data; and,
outputting from the AI algorithm a prediction score for the disease state in the test subject indicated by the data set obtained from the test subject, thereby generating the prediction score for the disease state in the test subject.

2. A computer-implemented method of generating a prediction score for a disease state in a test subject, the method comprising:
receiving, by a computer, a data set that comprises urine-related image data obtained from a test subject and/or one or more features extracted from the urine-related image data;
passing, by the computer, at least a portion of the data set through an artificial intelligence (AI) algorithm that is trained to generate one or more prediction scores for the disease state in subjects based at least in part on one or more portions of the data set; and,
outputting, by the computer, from the AI algorithm a prediction score for the disease state in the test subject indicated by at least the portion of the data set passed through the AI algorithm, thereby generating the prediction score for the disease state in the test subject.

3. The computer-implemented method of claim 1, wherein the AI algorithm has been trained on a first set of training data that comprises a plurality of sets of features extracted from urine-related image data obtained from reference subjects, wherein the urine-related image data obtained from the reference subjects are each labeled with a positive or negative disease state ground truth classification for a given reference subject, and wherein one or more predictions for a positive or negative disease state classification for the given reference subject are made based on the urine-related image data obtained from the given reference subject, which predictions are compared to the ground truth classification for the given reference subject when the AI algorithm is trained.

4. (canceled)

5. The computer-implemented method of claim 1, comprising generating a therapy recommendation for the test subject based upon the prediction score output from the AI algorithm.

6. The computer-implemented method of claim 1, comprising administering a therapy to the test subject based upon the prediction score output from the AI algorithm.

7.-8. (canceled)

9. The computer-implemented method of claim 1, wherein the AI algorithm is trained to generate prediction scores for the disease state in the subjects based at least in part on a set of features extracted from other types of data related to the subjects, and wherein the computer-implemented method further comprises passing a set of features extracted from the other types of data related to the test subject through the AI algorithm.

10. (canceled)

11. The computer-implemented method of claim 1, wherein the AI algorithm comprises at least first and second parts, wherein the first part is trained on a plurality of sets of features extracted from the urine-related image data related to the reference subjects, and wherein the second part is trained on a plurality of sets of features extracted from the other types of data related to the reference subjects.

12. The computer-implemented method of claim 1, wherein the other types of data comprise demographic data, symptom data, risk factor data, physical examination data, or a combination thereof.

13.-16. (canceled)

17. The computer-implemented method of claim 1, wherein the disease state comprises a bacterial infection, a viral infection, a fungal infection, a parasitic infection, hematuria, proteinuria, pyuria, glucosuria, or dehydration.

18.-23. (canceled)

24. The computer-implemented method of claim 1, wherein the images of urine samples obtained from the test and reference subjects are not magnified using a microscope.

25.-36. (canceled)

37. The computer-implemented method of claim 1 wherein the test subject obtains the images of the urine samples.

38.-43. (canceled)

44. A system for generating a prediction score for a disease state in a test subject using an artificial intelligence (AI) algorithm, the system comprising:
a processor; and
a memory communicatively coupled to the processor, the memory storing instructions which, when executed on the processor, perform operations comprising:
passing a data set that comprises one or more features extracted from urine-related image data obtained from a test subject through the AI algorithm that is trained to generate one or more prediction scores for the disease state in subjects based at least in part on the first set of features extracted from the urine-related image data; and
outputting from the AI algorithm a prediction score for the disease state in the test subject indicated by the data set obtained from the test subject.

45. The system of claim 44, wherein the instructions which, when executed on the processor, further perform operations comprising:
receiving the urine-related image data obtained from the test subject and/or one or more features extracted from the urine-related image data.

46. (canceled)

47. The system of claim 44, wherein the instructions which, when executed on the processor, further perform operations comprising:
generating a therapy recommendation for the test subject based upon the prediction score output from the AI algorithm.

48. The system of claim 44, wherein the AI algorithm has been trained on a first set of training data that comprises a plurality of sets of features extracted from urine-related image data obtained from reference subjects, wherein the urine-related image data obtained from the reference subjects are each labeled with a positive or negative disease state ground truth classification for a given reference subject, and wherein one or more predictions for a positive or negative disease state classification for the given reference subject are made based on the urine-related image data obtained from the given reference subject, which predictions are compared to the ground truth classification for the given reference subject when the AI algorithm is trained.

49.-53. (canceled)

54. The system of claim 44, wherein the AI algorithm comprises at least first and second parts, wherein the first part is trained on a plurality of sets of features extracted from the urine-related image data related to the reference subjects, and wherein the second part is trained on a plurality of sets of features extracted from the other types of data related to the reference subjects.

55. The system of claim 54, wherein the other types of data comprise demographic data, symptom data, risk factor data, physical examination data, or a combination thereof.

56.-66. (canceled)

67. The system of claim 44, wherein the images of urine samples obtained from the test and reference subjects are not magnified using a microscope.

68.-72. (canceled)

73. The system of claim 44, wherein the videos of the urine samples each comprise multiple views of the urine samples disposed in sample containers or other liquid receptacles.

74.-78. (canceled)

79. The system of claim 44, wherein the numerical vectors representing the set of parameterized symptoms from the reference subjects and from the test subject each comprise at least a 15-dimensional vector.

80.-81. (canceled)
